(19) United States
(12) Patent Application Publication
(10) Pub. No.: US 2006/0217303 A1
(43) Pub. Date: Sep. 28, 2006

(54) Compounds and Methods for Treating Seizure Disorders

(75) Inventor: Steven M. Kriegler, Madison, WI (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Wisconsin Alumni Research Foundation, Madison, WI (US)

(21) Appl. No.: 11/277,457

(22) Filed: Mar. 24, 2006

Related U.S. Application Data
(60) Provisional application No. 60/665,283, filed on Mar. 25, 2005.

(51) Int. Cl. A61K 38/26; A61K 31/70; A61K 31/57; A61K 31/19; A61K 31/203; A61K 31/202; A61K 31/265

(52) U.S. Cl. 514/12; 514/23; 514/177; 514/559; 514/560; 514/571; 514/557; 514/512

(57) ABSTRACT

This invention provides methods for alleviating seizure disorders in an animal, particularly epilepsy, by regulating the flux through the gluconeogenic enzyme PEPCK in brain cells.

This invention was made with government support under grant No. 025020 by the National Institutes of Health. The government has certain rights in the invention.

### BACKGROUND OF THE INVENTION

1. Field of the Invention

This invention relates to methods for alleviating seizure disorders in an animal. The invention particularly relates to relieving epilepsy, by regulating the rate of flux of substrate through the gluconeogenic enzyme phosphoenolpyruvate carboxykinase (PEPCK) and thereby regulating the cellular GTP to GDP ratio in brain cells. The invention specifically relates to the use of compounds that are alternative substrates to oxaloacetate for PEPCK, as anticonvulsant and antiepileptic agents for the treatment of seizures, epilepsy and other paroxysmal alterations in neurological and neuropsychiatric dysfunction. The invention also specifically relates to the use of compounds that regulate the flux of substrate through PEPCK by depleting the PEPCK reaction product, phosphoenolpyruvate (PEP), as anticonvulsant and antiepileptic agents for the treatment of seizures, epilepsy and other paroxysmal alterations in neurological and neuropsychiatric dysfunction. The invention also relates to the use of compounds that regulate the flux of substrate through PEPCK by increasing the amount of a PEPCK substrate, such as oxaloacetate, as anticonvulsant and antiepileptic agents for the treatment of seizures, epilepsy and other paroxysmal alterations in neurological and neuropsychiatric dysfunction. Further, the invention relates to alternative substrates for PEPCK., including glycolic acid, β-chloroacetate, L-glycerate or thioglycolate as anticonvulsant and antiepileptic agents for the treatment of seizures, epilepsy and other paroxysmal alterations in neurological and neuropsychiatric dysfunction

2. Background of the Invention

Functions of the central nervous system may be impaired by a variety of paroxysmal alterations including seizures, syncope, migraine, and transient ischemia. These alterations reflect a sudden, usually temporary interruption in some or all of the highly complex but organized function of nerve cells in the brain. Each individual has a “seizure threshold” or level of resistance to seizures: this threshold varies from person to person, most likely due to their genetic makeup and other developmental factors (Stafstrom, 1998, Pediatrics in Review 19: 335-344).

A person with a tendency to have repeated seizures may be suffering from epilepsy. Epilepsy is a generic term for a common serious neurological condition that affects one in every 200 adults and one in every 100 children (Hauser & Hersdorffer, 1990, EPILEPSY: FREQUENCY, CAUSES AND CONSEQUENCES, New York: Demos). Epilepsy is defined by recurrent episodes of seizures, which are brief involuntary behavioral alterations caused by paroxysmal intense electrical discharges in the brain. The causes of epilepsy are heterogeneous and include a diverse variety of genetic, metabolic, development, traumatic, neoplastic, and vascular etiologies which may present at any time from birth to senescence.

The diagnosis of epilepsy is based on clinical judgment, and may be supported by electroencephalogram, and in some cases, by MRI and blood tests. Seizures can be regarded as symptomatic manifestations of the underlying etiology or pathology. Epilepsy can sometimes be ameliorated by directly treating the underlying etiology, but anticonvulsant drugs, such as phenytoin, gabapentin, lamotrigine, felbamate, and topiramate, and others, which suppress the abnormal electrical discharges and seizures, are the mainstay of conventional treatment (Rho & Sankar, 1999, Epilepsia 40: 1471-1483). Currently available anticonvulsant drugs are effective in suppressing seizures in about 50% of patients, are moderately effective and reduce seizures in another 30-35%, and are ineffective in the remaining 15-20% of patients. The mechanisms of action of the currently-used aniticonviilsant drugs are complex and for the most part uncertain, but common general modes of anticonvulsant action include antagonism of sodium ion (Na⁺) channel function (which modifies repetitive use-dependent neuronal discharge), and modifications in γ-aminobutyric acid and glutamate-mediated synaptic transmission (which favorably alter the balance of excitation and inhibition in neural circuits). These drugs are also effective for treatment of other paroxysmal disorders including syncope, convulsive syncope, migraine, neuropathic pain, and neuropsychiatric conditions with paroxysmal or intermittent behavioral disturbances including bipolar disorders, affective disorders, anxiety disorders, stress disorders, and impulse disorders. In addition, anticonvulsants also provide neuroprotection and reduce infarct size in experimental models of stroke and ischemia.

Neurosurgery is an alternative treatment modality in a small proportion of people for whom drug treatment is ineffective. Patients who continue to have recurring seizures despite treatment with contemporary medications (˜50% of patients) are regarded as medically intractable, and a subset of these patients demonstrate progressive features such as increasing seizure frequency and cognitive decline. Patients with medically intractable epilepsy are usually considered for surgical resective treatment, which may be curative, when a localized irritative lesion can be identified. However, certain patients with intractable epilepsy are not candidates for surgical treatment because of the existence of multiple irritative lesions. This is especially true for children, for whom there is a subset that do not respond well with antiepileptic medications. For such patients, an alternative therapeutic modality is diet, specifically a high-fat diet known as the “ketogenic diet.” In many cases the ketogenic diet may produce effective and sometimes dramatic suppression of seizures and improvements in cognitive function.

The ketogenic diet has been employed for decades in children with epilepsy who have not adequately responded to medical therapy with conventional anticonvulsants (Wilder, 1921, Mayo Clinic Proceedings 2: 307-308; Freeman et al., 1998, Pediatrics 102: 1358-1363). The anticonvulsant action of the diet, which derives calories from high fat intake with very low or no carbohydrates and only adequate protein for growth, is associated with ketosis and production of the ketones β-hydroxybutyrate and acetoacetate. The ketogenic diet can be significantly efficacious and reduce seizures in a substantial subset of patients with severe epilepsy, but understanding of how the diet produces anticonvulsants effects has been limited. One of the remarkable features of the ketogenic diet is that the anticonvulsant effect develops during a period of at least days to weeks after beginning the diet, but is rapidly lost with intake of very minimal amounts of carbohydrate. Although the diet induces ketosis and generates ketone bodies (inter alia, β-hydroxybutyrate and acetoacetate), in experimental models ketone bodies are not consistently correlated with the anticonvulsant or anti-epileptic effects (Stafstrom & Bough, 2003, Nutritional Neuroscience 6: 67-79; Bough et al., 1999, Developmental Neuroscience 21: 400-406).

Despite its general efficacy, treating patients with the ketogenic diet, particularly children, has several drawbacks. Initiation of the diet typically requires hospitalization for up to one week, and the effects and benefits of the diet (i.e., seizure reduction) are usually not experienced immediately, being delayed from one week to three months from when the diet is started. Maintenance of the diet is difficult, since it requires a balance of nutrients at a particular ratio (usually 3:1 to 4:1 fats to all other nutrients) and intake of even a minimal amount of carbohydrates can eliminate the seizure-relieving benefits of the diet. Side-effects of the diet itself include nausea, vomiting, constipation, depression, sleepiness, lethargy, crankiness, decreased alertness, kidney stones, weight gain, increased serum cholesterol, and acidosis (Ballaban-Gil et al., 1998, Epilepsia 39: 744-748). In addition, the diet has limited effectiveness in adults, and can be even more difficult to implement with children who are allergic to dairy products.

Thus, there is a need in this art to develop methods and compounds for treating epilepsy, particularly medically-intractable epilepsy using alternatives to currently-available anti-epileptic drugs and neurosurgery. There is also a need to develop therapeutically-effective dietary methods other than the ketogenic diet that are easier to implement and maintain and that have fewer side effects and less severe consequences for non-compliance.

### SUMMARY OF THE INVENTION

This invention provides methods for alleviating seizure and paroxysmal disorders in an animal by regulating or altering the ratio of GTP to GDP in brain cells that provoke, initiate or maintain a seizure disorder. In particular, the invention provides methods for achieving the effect on GTP/GDP ratios by regulating the rate of flux through a gluconeogenic enzyme, phosphoenolpyruvate carboxykinase (PEPCK, E.C. 4.1.1.32), and thereby regulating the GTP/GDP ratio in cells involved in initiating, maintaining or perpetuating the seizure disorder in the animal. In preferred embodiments, the animal is a human, more preferably a human with epilepsy and most preferably adult or juvenile humans with medically-intractable or drug-resistant epilepsy.

The invention provides methods for treating a seizure disorder in an animal, comprising the step of administering an effective amount of a compound capable of regulating the rate of flux through PEPCK, to an animal in need thereof. In preferred embodiments, the compound is an alternative substrate for PEPCK, such as glycolic acid, β-chloroacetate, L-glycerate or thioglycolate. In alternative preferred embodiments, the compound reduces the concentration of the PEPCK, reaction product, usually phosphoenolpyruvate (PEP), such as 2-deoxy-D-glucose (2DG). In other preferred embodiments, the compound increases the concentration of a PEPCK substrate, such as oxaloacetate. In still further embodiments, the compound increases expression or activity of PEPCK in a cell. Preferably, the seizure disorder is epilepsy, most preferably medically-intractable or drug-resistant epilepsy. In a preferred embodiment, seizure frequency or occurrence are reduced by about 50%, more preferably by about 75% and most preferably by about 95%.

In certain additional embodiments, the methods provided by the invention reduce epileptic synchronous bursting in neural cells and in brain slices. In these embodiments, the methods comprise the step of contacting the cells with an effective amount of a compound capable of regulating the rate of flux through PEPCK. In preferred embodiments, the compound is an alternative substrate for PEPCK, such as glycolic acid, β-chloroacetate, L-glycerate or thioglycolate. In alternative preferred embodiments, the compound reduces the concentration of the PEPCK reaction product, usually phosphoenolpyruvate (PEP), such as 2-deoxy-D-glucose (2DG). In other preferred embodiments, the compound increases the concentration of a PEPCK substrate such as oxaloacetate. In still further embodiments, the compound increases expression or activity of PEPCK in a cell. In yet additional alternative embodiments, the method further comprises the step of contacting the cells with an amount of lactate or pyruvate sufficient to support metabolic integrity in the cells. Preferably, the neural cells are mammalian, more preferably human, and most preferably adult or juvenile human neural cells.

In alternative embodiments, the invention provides methods for alleviating seizure and paroxysmal disorders in an animal by increasing expression of PEPCK in brain cells that provoke, initiate or maintain a seizure disorder. In certain embodiments, PEPCK expression is increased by contacting the brain cell with corticosteroids such as dexamethasone, or with retinoic acid or derivatives thereof, or thyroid hormone. Pharmaceutical compositions of such compounds and methods for treating a seizure disorder by administering such compounds are also within the scope of this invention.

The invention also provides pharmaceutical compositions comprising oxaloacetate, alternative PEPCK substrates for PEPCK including but not limited to glycolic acid, β-chloroacetate, L,-glycerate or thioglycolate, and analogs thereof. The invention also provides pharmaceutical compositions comprising 2-deoxy-D-glucose (2DG), or related deoxy-substituted glucose compounds, such as 3-deoxy-D-glucose, 4-deoxy-D-glucose, 5-deoxy-D-glucose, combinations of other deoxy-glucose substitutions such as 2, n-deoxy-D-glucose (where n=3-5), compounds designated by permutations of the formula n, m deoxy-D-glucose (where n=2-5 and m=integers from 2-5 excluding n), sugars that can be metabolized into 2DG, such as 2-deoxy-D-galactose (which is metabolized into 2DG after phosphorylation to 2-deoxy-D-galactose-6-phosphate), and halogenated and other conjugated derivatives of deoxy sugars (as set forth above), such as fluoro-2-deoxy-D-glucose, conjugated deoxy sugars (as set forth above) that are metabolized to 2DG, formulated to be used according to the methods of the invention. The pharmaceutical compositions of the invention are provided formulated with pharmaceutically-acceptable excipients, adjuvants, or other components adapted to the mode of administration.

The methods of the invention are advantageous because they involve administration of compounds that are less toxic or that have fewer or more mild side-effects than the anticonvulsant and anti-epileptic drugs currently used to treat seizure disorders. The methods of the invention are also advantageous over dietary methods, such as the ketogenic diet known in the prior art, due to ease of implementation, easier and more likely compliance with their administration, less opportunity to avoid or neglect treatment compliance, smaller effects on serum lipids and cholesterol levels, less weight gain, more immediate effectiveness, and ease of monitoring. The inventive methods are advantageous as compared to neurosurgery in being less invasive and less irreversible.

Specific preferred embodiments of the present invention will become evident from the following more detailed description of certain preferred embodiments and the claims.

### DESCRIPTION OF THE DRAWINGS

An understanding of the invention is facilitated by reference to the drawings.

FIG. 1 is a schematic diagram of a portion of the chemical reactions and enzymatic mediators thereof occurring in gluconeogenesis in a mammalian cell, showing conversion of oxaloacetate to phosphoenolpynivate (PEP) by phosphoenolpyruvate carboxykinase (PEPCK) and the GTP energy requirement for the PEPCK reaction.

FIG. 2 illustrates the effects of alternative energy sources on epileptiform bursting frequency.

FIG. 2A is a series of electrophysiological traces of synchronized bursting in the CA3 area of rat hippocampal brain slices induced by increased potassium (K⁺) ion concentration, and illustrates a reduction in epileptic bursts produced by bath application of lactate (20 mM) as an alternative energy source. The top trace is an extracellular recording from the CA3 area of a hippocampal slice exposed to extracellular artificial cerebrospinal fluid (ACSF) solution containing 7.5 mM glucose for 1 hr. The middle trace is an extracellular recording from the CA3 area of a hippocampal slice following a change from glucose (10 mM) to lactate (20 mM) as the energy source in the media. The bottom trace is an extracellular recording from the CA3 area of the same hippocampal slice shown in the middle panel, after the energy source is changed back to glucose (10 mM).

FIGS. 2B and 2C are graphical representations demonstrating that using 20 mM lactate (FIG. 2B) and 20 mM pyruvate (FIG. 2C) as alternative cellular energy sources caused a reduction in epileptic bursts, and that reduction in epileptical bursting is lost when the alternative cellular energy source is removed from the ACSF.

FIGS. 3A and 3B are graphical representations demonstrating that the decrease in epileptical bursting caused by changing the energy source in the growth medium from glucose to lactate or pyruvate is mimicked when the brain slice is exposed to 10 mM glucose, 20 mM lactate and 1 mM 2-deoxyglucose (2DG) (FIG. 3A), or is exposed to 10 mM glucose, 20 mM lactate and 200 μM iodoacetate (FIG. 3B).

FIGS. 4A and 4B are graphical representations demonstrating that the addition of the PEPCK reaction product, PEP (5 mM), to the ACSF (FIG. 4A), or the addition of a PEPCK inhibitor, 3-mercaptopicolinic (3-MCP) (3 mM) (FIG. 4B), to the ACSF, resulted in an increase in epileptic bursting. FIGS. 4A and 4B also show that the increase in epileptical bursting caused by the addition of PEP or 3-MCP was lost when PEP or 3-MCP is removed from the ACSF.

FIGS. 5A and 5B are graphical representations demonstrating that decreased epileptic bursting caused by 2-DG or iodoacetate did not occur in brain slices simultaneously exposed to the PEPCK inhibitor 3-MCP.

FIG. 6A is a graphical representation demonstrating that the addition of excess substrate for PEPCK, oxaloacetate, or adding alternative substrates (glycolic acid, β-chloroacetate or thioglycolate), resulted in a decrease in epileptical bursting.

FIGS. 6B and 6C are graphical representations demonstrating that the decrease in epileptic bursting caused by the addition of excess oxaloacetate (FIG. 6B) or glycolic acid (FIG. 6C) substrates for PEPCK did not occur in brain slices simultaneously exposed to the PEPCK inhibitor 3-MCP.

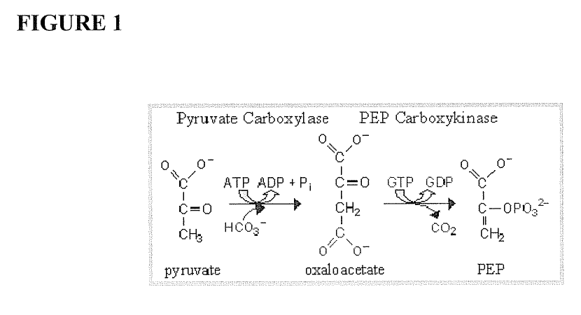

### DETAILED DESCRIPTION OF THE PREFERRED EMBODIMENTS

The invention provides methods and compounds for alleviating seizure disorders in an animal, particularly humans and including children having medically-intractable epilepsy. The methods provided by the invention relate to reducing seizures in an animal by regulating or altering the ratio of GTP to GDP in brain cells that provoke, initiate or maintain a seizure disorder. In particular, the invention provides methods for achieving the effect on GTP/GDP ratios by regulating the rate of flux through the gluconeogenic enzyme PEPCK. The methods of the invention specifically involve administering a therapeutically effective amount of a compound capable of regulating the rate of flux through PEPCK, and thereby regulating cellular GTP to GDP ratio to the animal. More particularly, the methods of the invention involve administering a compound which (a) serves as an alternative substrate for PEPCK., such as glycolic acid, β-chloroacetate, L-glycerate or thioglycolate; (b) reduces the concentration of PEPCK reaction products (e.g., PEP), such as 2-deoxyglucose or related compounds; or (c) increases the concentration of PEPCK substrates such as oxaloacetate, as set forth herein, in an amount effective to regulate the rate of flux through PEPCK, and thereby regulate the GTP to GDP ratio in brains of epileptic animals.

As used herein, the phrases “a compound capable of regulating the rate of flux through PEPCK,” or “a compound capable of regulating the rate of flux through the gluconeogenic enzyme PEPCK” are intended to encompass compounds that affect gluconeogenesis by altering the flux through PEPCK, particularly in brain cells involved in epileptic or synchronized bursting or in the brains of animals suffering from a seizure disorder, preferably humans and most preferably adult or juvenile humans with epilepsy. The phrases specifically encompass compounds that (a) serve as substrates alternative to, for example, oxaloacetate, including but not limited to glycolic acid, β-chloroacetate, L,-glycerate or thioglycolate; (b) reduce PEPCK reaction product concentration, for example phosphoenolpyruvate, including but not limited to 2-DG; (c) increase the amount or concentration of a PEPCK substrate such as oxaloacetate and (d) increase the amount or activity of PEPCK,. In preferred embodiments, a compound capable of regulating the flux through PEPCK of the invention is an alternative substrate for PEPCK, such as glycolic acid, β-chloroacetate, L-glycerate or thioglycolate. In another preferred embodiment, a compound capable of regulating the flux through PEPCK of the invention is 2-deoxyglucose, or a related deoxy-substitution of glucose, such as 3-deoxy-D-glucose, 4-deoxy-D-glucose, 5-deoxy-D-glucose, combinations of other deoxy-glucose substitutions such as 2, n-deoxy-D-glucose (where n=3-5), compounds designated by permutations of the formula n, in deoxy-D-glucose (where n=2-5 and m=integers from 2-5 excluding n). In additional preferred embodiments, a compound capable of regulating the rate of flux through PEPCK is a sugar that can be metabolized into 2DG, such as 2-deoxy-D-galactose (which is metabolized into 2DG after phosphorylation to 2-deoxy-D-galactose-6-phosphate), and halogenated and other conjugated derivatives of deoxy sugars (as set forth above), such as fluoro-2-deoxy-D-glucose, conjugated deoxy sugars (as set forth above) that are metabolized to 2DG, and compounds having antiglycolytic effects similar to 2DG, such as iodoacetate.

As used herein, the word “flux” when applied as in the “flux of substrate through PEPCK” is intended to describe the amount or flow of a molecule through a reaction catalyzed by PEPCK. The term is intended to describe a dynamic process regulated by cellular factors including the amount or concentration of substrates, reaction products, and co-factors; intracellular location of substrates, reaction products and co-factors; and the amounts or concentrations of the substrates, reaction products and co-factors of other components of the glycolytic or glyconeogenic pathways in a cell that control or influence reactions catalyzed by PEPCK. The term is also intended to encompass expression or activity levels of PEPCK itself, caused or as the result of changes in transcription or translation of the PEPCK gene, or changes in degradation of PEPCK protein, or changes in PEPCK substrate affinity, enzymatic activity or turnover rate. In certain embodiments, compounds that increase PEPCK expression and thereby increase the flux of substrate through PEPCK (and thus change the GTP to GDP ratio in the cell) is glucagon, long-chain unsaturated fatty acids and oleate. In alternative embodiments, the compound is dexamethasone, clofibrate, isoprenaline or retinoic acid. In yet further alternative embodiments, the compound is a peroxisome proliferators-activated receptor (PPAR) agonist. In the latter embodiments, one having ordinary skill will recognize that certain PPAR receptor subtypes will increase while others will decrease PEPCK expression, thereby providing a sensitive capacity for modulating PEPCK expression. In preferred embodiments, the PEPCK expression inhibitor includes fibric acid derivatives including but not limited to commercially-available pharmaceutical agents, such as Gemfibozil (Lopid®), Fenofibrate (Tircor®) and Clofibrate (Atromid-S®). See Antras-Ferry et al., 1995, Eur. J Biochem. 234: 390-396.

As used herein, the term “seizure disorders” includes but is not limited to infantile spasms, myoclonic and “minor motor” seizures, as well as tonic-clonic seizures and partial complex seizures. In preferred embodiments, the seizure disorder is epilepsy, including idiopathic, symptomatic and cryptogenic epilepsy, and more preferably drug-resistant or medically-intractable epilepsy, by which is meant that epileptic seizures continue despite adequate administration of antiepileptic drugs.

As used herein, the term “paroxysmal disorders” includes syncope, convulsive syncope, migraine, neuropathic pain, tics, tremors and other movement disorders, and neuropsychiatric conditions with paroxysmal or intermittent behavioral disturbances including bipolar disorders, affective disorders, anxiety disorders, and stress disorders.

As used herein, the term “juvenile,” particularly when applied to a human patient is a human less than 18 years old, more preferably less than 16 years old, more preferably less than 14 years old, more preferably less than 12 years old, most preferably less than 10 years old.

As used herein, the term “ketogenic diet” is intended to describe low carbohydrate, high fat diets used as an alternative to drug therapy for epilepsy in children. In the “classic” form of the diet, calories are provided from food naturally high in fats, such as cream, cheese, mayonnaise, butter and oil. In this form, the proportion of fats to carbohydrates and protein in the diet is about 4:1 (by weight, equivalent to a 9:1 ratio by caloric content). In an alternative form, the diet is supplemented with medium chain triglycerides (MCT). The ketogenic diet has been employed for decades in children with epilepsy who have not adequately responded to medical therapy with conventional anticonvulsants. The anticonvulsant action of the diet, which derives calories from high fat and protein intake with very low or no carbohydrates, is associated with ketosis and production of the ketones 0-hydroxybutyrate and acetoacetate. The “ketogenic” diet can be significantly efficacious and reduce seizures in a substantial subset of patients with severe epilepsy, but understanding of how the diet produces anticonvulsant effects is limited. One of the remarkable features of the ketogenic diet is that the anticonvulsant effect is rapidly lost with intake of very minimal amounts of carbohydrate. Most research has focused oil the role of ketone bodies for the anti-epileptic effect of the diet, but has not addressed the observed peculiarity that the anticonvulsant effects of the diet are rapidly lost with minimal carbohydrate intake.

As used herein, “antiepileptic drugs” include but are not limited to gabapentin (Neurontin®), carbamazepine (Tegretol®), ethosuximide (Zarontin®), lamotrigine (Lamictal®), felbamate (Felbatol®), topiramate (Topamax®), zonisamide (Zonergran®), tiagabine (Gabitril®), oxcarbazepine (Trileptal®), levetiracetam (Keppra®), divalproex sodium (Depakote®), phenytoin (Dilantin®), fos-phyenytoin (Cerebryx®).

As used herein, an “effective amount” or “therapeutically effective amount” of a compound capable of regulating the rate of flux through PEPCK is defined as an amount that when administered to an animal, preferably a human, more preferably a human having a seizure disorder including both adults and juvenile humans with epilepsy, reduces the frequency, duration or severity of seizures experienced by the individual. The “effective amounts” of said compounds capable of regulating the rate of flux through PEPCK will depend on species, pharmacokinetics, and route of administration.

As used herein the term “metabolic integrity ” is intended to mean that the cell is viable and metabolically active, and specifically is not apoptotic or metabolically impaired by existence in a low glucose environment. The term in particular is intended to mean that the energy balance of the cell and its capacity to meet its normal energetic requirements is maintained.

Glycolysis is the metabolic pathway for obtaining energy from glucose. The utilization of glucose as an energy source requires entry into the cell by specific hexose transporters, including but not limited to GLUT1 (SLC2A1, Accession Number AC023331), GLUT2 (SLC2A2, AC068853), GLUT3 (SLC2A3, AC007536), GLUT4 (SLC2A4, AC003688), GLUT5 (SLC2A5, AC041046), GLUT6 (SLC2A6, AC002355), GLUT7 (SLC2A7, AL356306), GLUT8 (SLC2A8, AL445222), GLUT9 (SLC2A9, AC005674), GLUT10 (SLC2A10, AC031055), GLUT11 (SLC2A11, AP000350), GLUT11 (SLC2A11, AP000350), GLUT12 (SLCA12, ALA49363), or GLUT13 (SLCA13, AJ315644). After entry into the cell, glucose is phosphorylated to form 6-phospho-glucose (6-P-G); this phosphorylation is performed by hexokinases, which are expressed ubiquitously in mammalian tissues, and glucokinases, which are expressed in liver and in some brain cells. 6-P-G is then isomerized to form 6-phospho-fructose by phosphoglucose isomerase (E.C. 5.3.1.9). This reaction requires the opening of the 5-carbon glucose ring followed by closure to form a 4 carbon ring, which occurs by oxidation of the 2 carbon hydroxyl group to a keto group. 6-phospho-fructose is in turn phosphorylated to 1,6 diphosphofructose by 6-phosphofructose-1-kinase (E.C. 2.7.1.1 1), and this compound is cleaved to glyceraldehyde-3-phosphate and dilhydroxyacetone phosphate by fructose bisphosphate aldolase (E.C. 4.1.2.13). The dihydroxyacetone phosphate formed in this reaction is converted to glyceraldehyde-3-phosphate, which is the substrate for glyceraldehyde-3-phosphate dehydrogenase (E.C. 1.2.1.12), forming 1,3 phosphoglycerate. 1,3 phosphoglycerate is converted to 3-phosphoglycerate by 3-phosphoglycerate kinase (E.C. 2.7.2.3), and the 3-phosphoglycerate product of this reaction is converted to 2-phosphoglycerate by phosphoglyceromutase (E.C. 5.4.2.1). The enzyme enolase (E.C. 4.2.1.11) converts 2-phosphoglycerate to phosphoenol pyruvate (PEP), which then forms pyruvate by the action of pynivate kinase (E.C. 2.7.1.40). Pyruvate can then be converted to lactate or acetyl-CoA, depending on metabolic conditions in the cell. As provided herein, inhibition of glycolysis after PEP is produced is not expected to increase flux through PEPCK and thus is expected to be ineffective.

Antiglycolytic compounds, that is, compounds that reduce glucose metabolism, such as 2DG and iodoacetate, have been shown to be effective in reducing epileptic or synchronized bursting in the brains of animals suffering from a seizure disorder (as disclosed in co-owned and co-pending U.S. Ser. No. 60/580,436, filed Jun. 17, 2004, the disclosure of which is explicitly incorporated by reference herein.

Blood glucose levels in animals or humans who are calorie restricted or on the ketogenic diet are maintained in the normal range primarily through gluconeogenesis in the liver.

Gluconeogenesis is the metabolic pathway responsible for biosynthesis of new glucose. Gluconeogenesis shares many enzymes with the glycolytic pathway, however, three reactions of glycolysis have such a large negative ΔG in the forward direction that they are essentially irreversible: hexokinase, phosphofructokinase, and pyruvate kinase. Thus, these steps must be bypassed in gluconeogenesis. The starting point for the gluconeogenesis pathway is the conversion of pyruvate to oxaloacetate by the enzyme pyruvate carboxylase (E.C. 6.4.1.1). The conversion to oxaloacetate requires the input of energy in the form of ATP. The oxaloacetate product is converted to phosphoenolpyruvate (PEP) by phosphoenolpyruvate carboxykinase (PEPCK; E.C. 4.1.1.32) in a reaction requiring the input of energy in the form of GTP. The PEP formed in this reaction is converted to 2-phosphoglycerate by enolase (E.C. 4.2.1.11), and the 2-phosphoglycerate product of this reaction is converted to 3-phosphoglycerate by phosphoglyceromutase (E.C. 5.4.2.1). The 3-phosphoglycerate formed in this reaction is converted to 1,3-bisphosphoglycerate by phosphoglycerate kinase (E.C. 2.7.2.3) in a reaction requiring the input of energy in the form of ATP. The 1,3-bisphosphoglycerate formed in this reaction is converted to fructose-1,6-bisphosphate by two enzymes: glyceraldehyde-3-phosphate dehydrogenase (E.C. 1.2.1.12), in a reaction that requires NADH, and aldolase (E.C. 4.1.2.13). The fructose-1,6-bisphosphate formed in this reaction is converted to fructose-6-phosphate by fructose-1,6-bisphosphatase (E.C. 3.1.3.11), and fructose-6-phosphate product of this reaction is converted to glucose-6-phosphate by phosphoglucose isomerase (E.C. 5.3.1.9). Finally, glucose-6-phosphate product of this reaction is converted to glucose by glucose-6-phosphatase (E.C. 3.1.3.9). The role of gluconeogenesis in the brain is less well understood, but it is clear that the brain as a whole can be gluconeogenic (Bhattacharya & Datta, 1993, Mol Cell Biochem. 125: 51-7; Ghosh et al., 2005, J Biol Chem. 10.1074/jbc.M410894200, posted Jan. 20, 2005 on www.jbc.org, lasted visited Mar. 1, 2005) and PEPCK is expressed in glial cells and neurons (Cruz et al., 1998, J. Neurochem. 70:2613-9). However, NMR studies suggest that most of the actual glucose production occurs in the glial metabolic pool (Schmoll et al., 1995, Eur J. Biochem. 227: 308-15). Thus, the role of PEPCK in neurons may be significantly different than other gluconeogenic cell types.

The compounds provided by the invention, and methods for using them as anticonvulsants and anti-epileptic agents, regulate the rate of flux through the PEPCK, and as a consequence thereby regulate the cellular GTP to GDP ratio. In preferred embodiments, compounds capable of regulating the rate of flux through PEPCK are alternative substrates for PEPCK, including but not limited to glycolic acid, β-chloroacetate, L-glycerate or thioglycolate. In alternative preferred embodiments, 2-DG and related compounds (such as halogenated derivatives like 2-fluoro-deoxyglucose-D-glucose, or other deoxy derivatives of hexose sugars including 2-deoxy galactose, which function in a analogous manner and prevent galactose from being used as a carbon source, and 3-deoxy-D-glucose, 4-deoxy-D-glucose, 5-deoxy-D-glucose, or combinations of other deoxy-glucose substitutions such as 2, n-deoxy-D-glucose (where n=3-5), compounds designated by permutations of the formula 71, m deoxy-D-glucose (where n=2-5 and m=integers from 2-5 excluding n), sugars that can be metabolized into 2DG, such as 2-deoxy-D-galactose (which is metabolized into 2DG after phosphorylation to 2-deoxy-D-galactose-6-phosphate), and halogenated and other conjugated derivatives of deoxy sugars (as set forth above), such as fluoro-2-deoxy-D-glucose, conjugated deoxy sugars (as set forth above) that are metabolized to 2DG) regulate flux through PEPCK by reducing the concentration of the usual PEPCK reaction product, phosphoenolpyruvate (PEP). In other preferred embodiments, compounds capable of regulating the rate of flux of through the gluconeogenic enzyme PEPCK, increase the concentration of a PEPCK, substrate PEPCK, oxaloacetate. Cellular oxaloacetate concentration is tightly controlled by the NADH/NAD+ ratio, and changing this ratio changes gluconeogenesis (Sistare & Hayse 1985, J Biol. Chem. 260: 12748-12753).

The present invention specifically provides glycolic acid, β-chloroacetate, L,-glycerate or thioglycolate, 2-deoxy-D-glucose (2DG) and pharmaceutical formulations thereof as anticonvulsant and antiepileptic agents for treating seizures, epilepsy and other paroxysmal alterations in neurological and neuropsychiatric dysfunction. This invention includes 2DG and related deoxy-substitutions of glucose (as described above), halogenated derivatives and conjugates of these compounds that also regulate the rate of flux through PEPCK, and thereby regulate the cellular GTP to GDP ratio, sugars such as 2-deoxy-D-galactose and other compounds that are metabolized into 2DG and act in the central nervous system by regulating the rate of flux through PEPCK, and thereby regulate the cellular GTP to GDP ratio and compounds modifying reactions in other metabolic pathways that mimic the effects of regulating the rate of flux through PEPCK, and thereby regulate the cellular GTP to GDP ratio on those pathways and have anticonvulsant and antiepileptic effects.

As disclosed herein, oxaloacetate, glycolic acid, β-chloroacetate, L-glycerate or thioglycolate, iodoacetate, and 2DG were effective against epileptic discharges evoked in vitro by decreasing the relative burst frequency under normal glucose conditions. Compounds that can substitute for the usual substrate of PEPCK, oxaloacetate, regulate the rate of flux through PEPCK, and thereby regulate the cellular GTP to GDP ratio by serving as additional substrate sources for the enzyme. 2DG acts in the central nervous system by inhibiting glycolysis, for example by regulating the rate of flux through PEPCK, and thereby regulating the cellular GTP to GDP ratio at by reducing the concentration of the PEPCK reaction product, PEP. Compounds that regulate glycolysis and gluconeogenesis can also have associated effects on other metabolic pathways that may cumulatively influence energy generation, intracellular signaling pathways, and long-term regulation of cellular function, making these compounds useful treatments for paroxysmal alterations in neurological and neuropsychiatric function such as seizures, epilepsy, migraine, syncope, neuropathic pain, anxiety, and mood disorders.

2DG is known in the art and itself and derivatives thereof have been used medicinally, particularly as a radiolabeled tracer molecule in positron emission tomography (PET) scans of myocardium for diagnosing ischemic heart disease and brain seizures in humans, as well as certain malignancies (see www.fda.gov/cder/regulatory/pet/fdgoincologyfinal.htm, visited Dec. 23, 2003). 2DG has also been used as a chemotherapeutic agent against breast cancer (Kaplan et al., 1990, Cancer Research 50: 544-551).

Glycolic acid and glycerate have been used in cosmetics and skin creams, and have been detected in humans as the metabolites of other medicines

The invention also provides embodiments of compounds that regulate the rate of flux through PEPCK, and thereby regulate the cellular GTP to GDP ratio at as pharmaceutical compositions. The pharmaceutical compositions of the present invention can be manufactured in a manner that is itself known, e.g., by means of a conventional mixing, dissolving, granulating, dragee-making, levigating, emulsifying, encapsulating, entrapping or lyophilizing processes.

Pharmaceutical compositions of the compounds of the present invention that regulate the rate of flux through PEPCK, and thereby regulate the cellular GTP to GDP ratio can be formulated and administered through a variety of means, including systemic, localized, or topical administration. Techniques for formulation and administration can be found in “Remington's Pharmaceutical Sciences,” Mack Publishing Co., Easton, Pa. The mode of administration can be selected to maximize delivery to a desired target site in the body. Suitable routes of administration can, for example, include oral, rectal, transmucosal, transcutaneous, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the compounds of the present invention that regulate the rate of flux through PEPCK, and thereby regulate the cellular GTP to GDP ratio in a local rather than systemic manner, for example, via injection of the compound directly into a specific tissue, often in a depot or sustained release formulation.

Pharmaceutical compositions for use in accordance with the methods of the present invention thus can be formulated in conventional manner using one or more physiologically acceptable carriers comprising excipients and auxiliaries that facilitate processing of antiglycolytic compounds into preparations that can be used pharmaceutically. Proper formulation is dependent upon the route of administration chosen.

The compounds of the present invention that regulate the rate of flux through PEPCK, and thereby regulate the cellular GTP to GDP ratio at can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the compounds of the present invention that regulate the rate of flux through PEPCK, and thereby regulate the cellular GTP to GDP ratio can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

For injection, compounds of the present invention that regulate the rate of flux through PEPCK, and thereby regulate the cellular GTP to GDP ratio can be formulated in appropriate aqueous solutions, such as physiologically compatible buffers such as Hank's solution, Ringer's solution, lactated Ringer's solution, or physiological saline buffer. For transmucosal and transcutaneous administration, penetrants appropriate to the barrier to be permeated are used in the formulation. Such penetrants are generally known in the art.

For oral administration, compounds of the present invention that regulate the rate of flux through PEPCK, and thereby regulate the cellular GTP to GDP ratio can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, antiglycolytic compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation compounds of the present invention that regulate the rate of flux through PEPCK, and thereby regulate the cellular GTP to GDP ratio are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

In addition to the formulations described previously compounds of the present invention that regulate the rate of flux through PEPCK, and thereby regulate the cellular GTP to GDP ratio can also be formulated as a depot preparation. Such long acting formulations can be administered by implantation (for example subcutaneously or intramuscularly) or by intramuscular injection. Thus, for example, the antiglycolytic compounds can be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for hydrophobic embodiments of the compounds of the present invention that regulate the rate of flux through PEPCK, and thereby regulate the cellular GTP to GDP ratio is a co-solvent system comprising benzyl alcohol, a nonpolar surfactant, a water-miscible organic polymer, and an aqueous phase. The co-solvent system can be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g. polyvinyl pyrrolidone; and other sugars or polysaccharides can substitute for dextrose.

Alternatively, other delivery systems can be employed. Liposomes and emulsions are well known examples of delivery vehicles or carriers for hydrophobic drugs. Certain organic solvents such as dimethylsulfoxide also can be employed, although usually at the cost of greater toxicity. Additionally, compounds of the present invention that regulate the rate of flux through PEPCK, and thereby regulate the cellular GTP to GDP ratio can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the compounds of the present invention that regulate the rate of flux through PEPCK, and thereby regulate the cellular GTP to GDP ratio for a few weeks up to over 100 days.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

The invention also provides formulations of the compounds of the present invention that regulate the rate of flux through, and thereby regulate the cellular GTP to GDP ratio as foodstuffs, food supplements or as a component of a food for an animal, preferably a human, more preferably a human with epilepsy and most preferably adult or juvenile humans with medically-intractable or drug-resistant epilepsy.

For any compounds of the present invention that regulate the rate of flux through PEPCK, and thereby regulate the cellular GTP to GDP ratio used in the method of the invention, the therapeutically effective dose can be estimated initially from in vitro assays, as disclosed herein, or using art-recognized animal model systems or a combination thereof. For example, a dose can be formulated in animal models to achieve a circulating concentration range that includes the EC₅₀ (effective dose for 50% increase) as determined in vitro, i.e., the concentration of the test compound which achieves a half-maximal amount of seizure frequency. Such information can be used to more accurately determine useful doses in humans.

It will be understood, however, that the specific dose level for any particular patient will depend upon a variety of factors including the activity of the antiglycolytic compounds employed, body weight, general health, sex, diet, time of administration, route of administration, and rate of excretion, drug combination, the severity and extent of the particular seizure disorder in the patient undergoing therapy and the judgment of the prescribing physician and in particular the age of the patient, who is preferably a juvenile and more preferably pre-pubescent.

Preferred compounds of the present invention that regulate the rate of flux through PEPCK, and thereby regulate the cellular GTP to GDP ratio provided by the invention will have certain pharmacological properties. Such properties include, but are not limited to oral bioavailability, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcová et al. (1996, J. Chromat. B 677: 1-27). In vitro half-lives of compounds of the present invention that regulate the rate of flux through PEPCK, and thereby regulate the cellular GTP to GDP ratio may be predicted from assays of microsomal half-life as described by Kuhnz and Gieschen (1998, Drug Metabolism and Disposition, 26: 1120-1127).

Toxicity and therapeutic efficacy of the compounds of the present invention that regulate the rate of flux through PEPCK, and thereby regulate the cellular GTP to GDP ratio can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds of the present invention that regulate the rate of flux through PEPCK, and thereby regulate the cellular GTP to GDP ratio that exhibit high therapeutic indices are preferred. The data obtained from these cell culture assays and animal studies can be used in formulating a range of dosage for use in humans. The dosage of such compounds of the present invention that regulate the rate of flux through PEPCK, and thereby regulate the cellular GTP to GDP ratio lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch. 1, p. 1).

Dosage amount and interval of administration of compounds of the present invention that regulate the rate of flux through PEPCK, and thereby regulate the cellular GTP to GDP ratio can be adjusted individually to reduce seizure frequency, duration or intensity For example, doses of 250 mg/kg 2DG or less to higher as tolerated can be used to reduce seizure frequency and minimize toxicity. Doses of 650 mg/kg 2DG are well tolerated in rats. Glycolic acid has an LD₅₀ of ˜1900 mg/kg; 5 mM solutions of oxaloacetate glycolic acid, chloroacetate and thioglycolate are equivalent to 660 mg/kg 380 mg/kg, ˜620 mg/kg and 460 mg/kg. respectively. The anticonvulsant effects of 2DG administered at 250 mg/kg twice daily for 3 months lasted for approximately 8 weeks after stopping 2DG while continuing twice daily stimulation, indicating that effects of 2DG are quite prolonged. A practitioner skilled in the art can adjust dosage in the range up to 500-600 mg/kg 2DG and the timing of administration to produce prolonged anticonvulsant and antiepileptic effects. Efficacious dosage amounts can be adjusted to about 14 mg/kg 2-DG in children and 40 mg/kg 2-DG in adults, using therapeutic efficacy measurements (e.g., reduction in frequency or severity of seizures) as a criterion for establishing effective dosage levels.

For the embodiments such as compounds of the present invention that regulate the rate of flux through PEPCK, and thereby regulate the cellular GTP to GDP ratio by providing alternative substrates for PEPCK, dosage amount and timing of administration of said compounds can be adjusted individually to provide plasma levels of the compounds of the present invention which are sufficient to reduce seizure frequency, duration or intensity.

For the embodiments such as compounds of the present invention that regulate the rate of flux through PEPCK, and thereby regulate the cellular GTP to GDP ratio by reducing the concentration of the reaction products of PEPCK, dosage amount and timing of administration of said compounds can be adjusted individually to provide plasma levels of the compounds of the present invention which are sufficient to reduce seizure frequency, duration or intensity.

For the embodiments such as compounds of the present invention that regulate the rate of flux through PEPCK, and thereby regulate the cellular GTP to GDP ratio by increasing the amount of the usual substrate for PEPCK, oxaloacetate, dosage amount and timing of administration of said compounds can be adjusted individually to provide plasma levels of the compounds which are sufficient to reduce seizure frequency, duration or intensity.

The invention provides methods for reducing seizure frequency, duration or intensity in an animal, preferably an adult or juvenile human. The methods of the invention are effective for reducing seizure frequency, duration or intensity in at least 50%, more preferably 60%, more preferably 70%, more preferably 80%, more preferably 90%, more preferably 95%, more preferably 98%, and more preferably 99% of treated patients. In preferred embodiments, the inventive methods are practiced using the pharmaceutical compositions of the invention as disclosed herein.

The Examples which follow are illustrative of specific embodiments of the invention, and various uses thereof. They set forth for explanatory purposes only, and are not to be taken as limiting the invention.

### EXAMPLE 1

### Effect of Energy Source on Synchronized Bursting in Hippocampal Slices

The effect of various energy sources on synchronized bursting induced by elevation of [K⁺]ₒ in rat hippocampal slices ex corpora was evaluated.

In these experiments, postnatal day 28 to 40 male Sprague-Dawley rats were anesthetized and decapitated. Brains were removed and transferred to ice cold artificial cerebrospinal fluid (ACSF, comprising 124 mM NaCl, 5 mM KCl, 1.25 mM NaH₂PO₄, 1.5 mM MgSO₄, 26 mM NaHCO₃ and 2 mM CaCl₂), supplemented with 10 mM glucose, which was continuously bubbled with 95% O₂ and 5% CO₂. Transverse hippocampal slices (˜500 microns) were prepared on a Leica VT1000s vibratome (Wetzlar Germany). The slices were allowed to recover for 1 hour at room temperature and were then transferred to an interface recording chamber at 34° C. in ACSF with 7.5 mM [K⁺]ₒ. Extracellular recordings were made from the CA3 region with an Axioclamp 2B (Axon Instruments, Forest City, Calif.) using a glass microelectrode filled with 150 mM NaCl. Data were recorded and analyzed using PClamp8 (Axon Instruments).

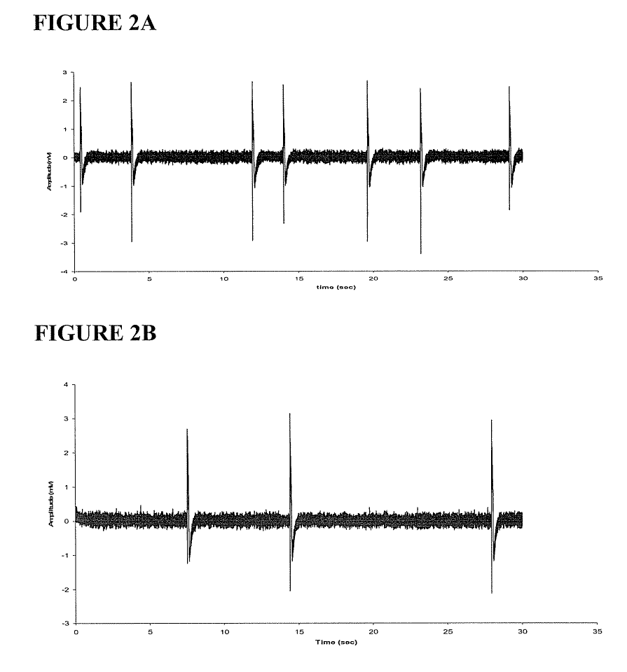

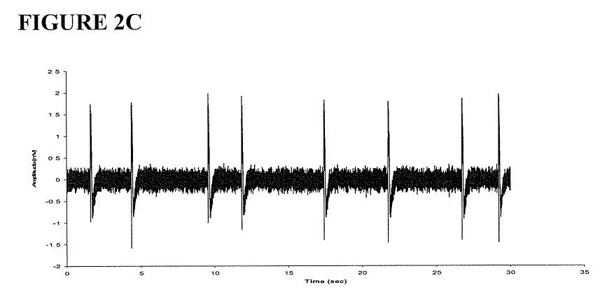

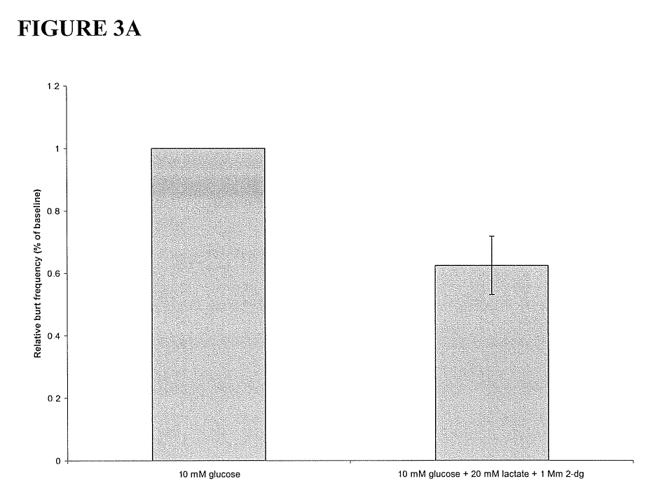

Synchronized bursting was induced by incubating hippocampal slices in ACSF supplemented with potassium chloride to a final concentration of 7.5 mM [K⁺]ₒ. Baseline recordings were obtained after exposure to elevated [K⁺]ₒ for 1 hour and the burst frequency had stabilized. Bursting was then recorded in ACSF containing 20 mM lactate or 20 mM pyruvate in place of the 10 mM glucose. The results of these experiments are shown in FIGS. 2A through 2C. The burst frequency reversibly decreased to 63±8% of the baseline after addition of lactate as shown graphically in FIG. 2B. As shown in the upper trace in FIG. 2A, the average burst frequency at baseline in 10 mM glucose was found to be regular. The middle trace in FIG. 2A shows that the average burst frequency increases when the slice is exposed to 20 mM lactate, and this effect is reversible when the lactate is replaced with 10 mM glucose (FIG. 2A, bottom trace). The burst frequency reversibly decreased to 4±2% of the baseline after addition of pyruvate as shown graphically in FIG. 2C. These results demonstrated that removal of glucose and substitution with alternative energy sources such as lactate or pyruvate suppress synchronized bursts in CA3 and have anticonvulsant effects.

### EXAMPLE 2

### Reduction of Synchronized Bursting by 2DG and Iodoacetate

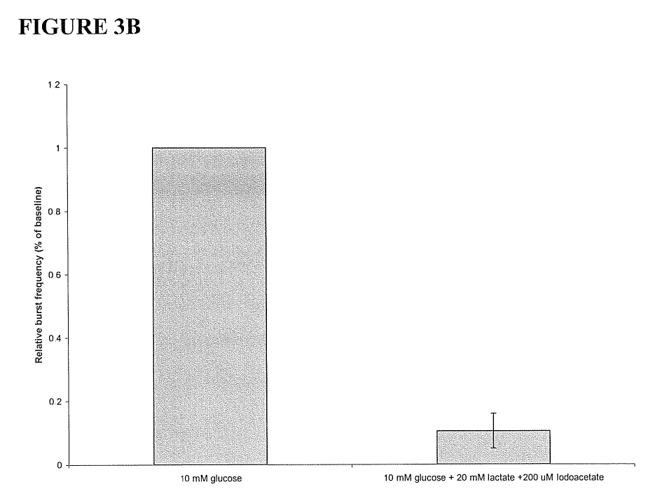

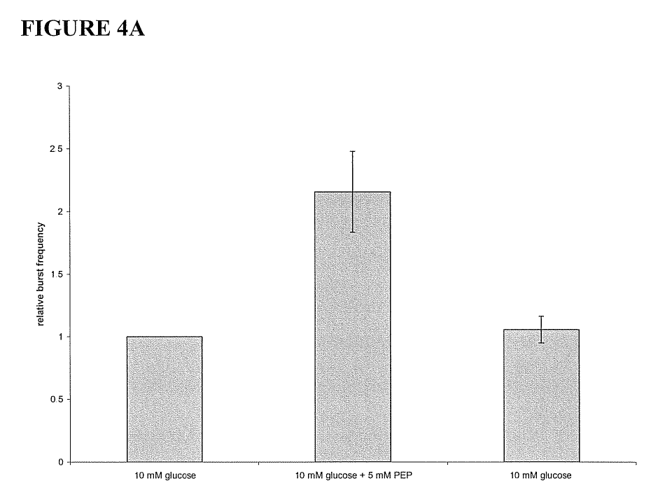

The antiepileptic effect of replacing glucose was compared to the impact of chemically inhibiting glycolysis. The experiments set forth in Example 1 were repeated using ACSF supplemented with 20 mM lactate in the presence of 1 mM 2DG or 200 μM iodoacetate, an inhibitor of the glycolytic enzyme glyceraldehyde phosphate dehydrogenase (EC 1.2.1.12). The results of these experiments are shown in FIGS. 3A and 3B. FIG. 3A shows the rate of baseline synchronized bursting from a hippocampal slice in ACSF with 10 nM [K⁺]ₒ 10 mM glucose. 2DG (in the presence of 20 mM lactate) reduced synchronized bursting. FIG. 3B shows the rate of baseline synchronized bursting from a hippocampal slice in ACSF with 10 mM [K⁺]ₒ 10 mM glucose. Iodoacetate also reduced synchronized bursting. The results with 2DG and iodoacetate demonstrate that inhibiting glycolysis is an effective means for reducing neural synchronization, the cellular event associated with various seizure disorders.

### EXAMPLE 3

### Induction of Synchronized Bursting by Alteration of PEPCK Activity

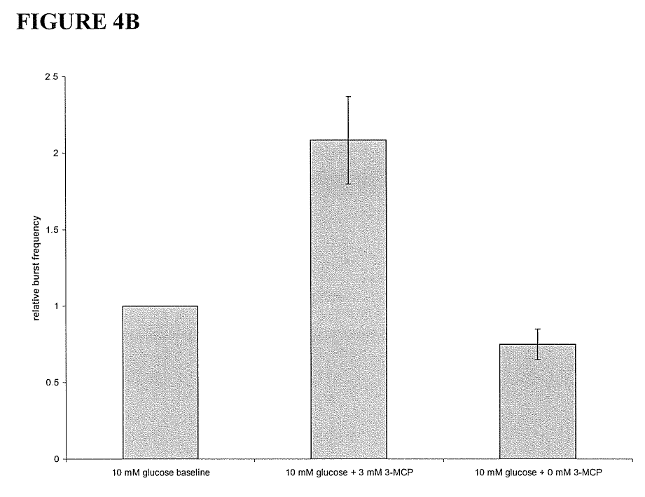

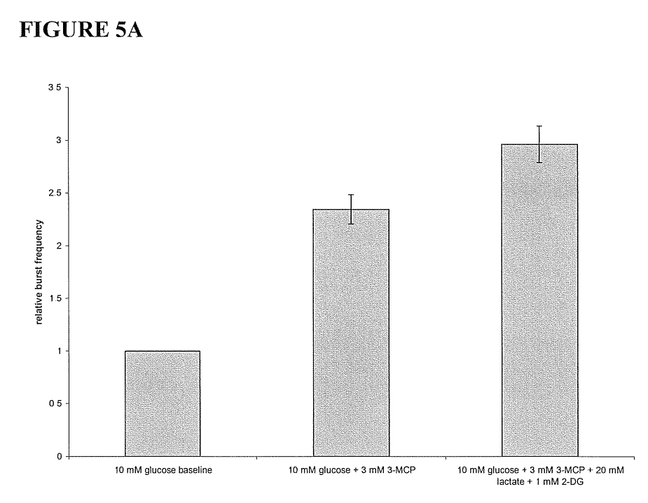

To further understand the mechanism of the antiepileptic effect of the ketogenic diet, another pathway that could be activated by the diet, the gluconeogenic pathway, was studied. Specifically, regulation of a GTP-dependent enzyme in the gluconeogenic diet, PEPCK, was studied for the effect on epileptiform bursting. The experiments set forth in Example 1 were repeated using ACSF supplemented with 10 mM glucose in the presence of 5 mM PEP, the reaction product of PEPCK, or in the presence of 3 mM 3-mercaptopicolinic acid (3-MCP), a specific inhibitor of PEPCK. As shown graphically in FIG. 4A, inhibition of PEPCK by addition of the reaction product, PEP, reversibly activated the burst frequency of brain slices, more than doubling the rate of bursting (215±32% of baseline). Similarly, as shown in FIG. 4B, the specific inhibitor of PEPCK, 3-MCP, also greatly increase the burst frequency of brain slices, again more than doubling the rate (208±28% of baseline). Inhibiting PEPCK dramatically affected burst frequency in brain slices, indicating that PEPCK is important component in the regulation of bursting in hippocampal cells.

### EXAMPLE 4

### Effect of Inhibition of PEPCK on Synchronized Bursting in Hippocampal Slices in the Presence of 2DG or Iodoacetate

To investigate whether the gluconeogenic enzyme PEPCK is important for the antiepileptic effects of reducing glucose utilization, the effect of the specific PEPCK inhibitor 3-MCP on 2DG- and iodoacetate-induced decreases in burst frequency were tested.

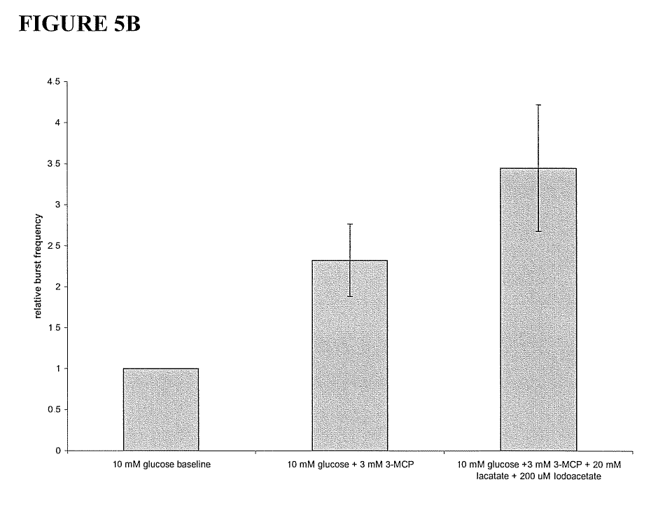

The effects of glucose deprivation on synchronized burst discharges were examined in rat hippocampal slices ex corpora using the methods described in Example 1. FIGS. 5A and 5B confirm that 3-MCP induces an increase in the rate of bursting. The increase in bursting caused by the specific PEPCK inhibitor could not be blocked by either 2DG (FIG. 5A) or iodoacetate (FIG. 5B), even though these compounds alone cause significant decreases in bursting (see FIG. 3). Thus, PEPCK enzyme activity is dominant over the antiepileptic and anticonvulsant effects of 2DG and iodoacetate.

### EXAMPLE 5

### Effect of Substrate Flux through PEPCK on Synchronized Bursting in Hippocampal Slices

To confirm that substrate flux through the gluconeogenic enzyme PEPCK is antiepileptic, rather than changes “downstream” of PEPCK, (i.e., towards the pathway end-product, glucose) in the gluconeogenic pathway, alternative substrates for PEPCK were tested that are catalyzed in the same direction as oxaloacetate to PEP, and which also require GTP energy input.

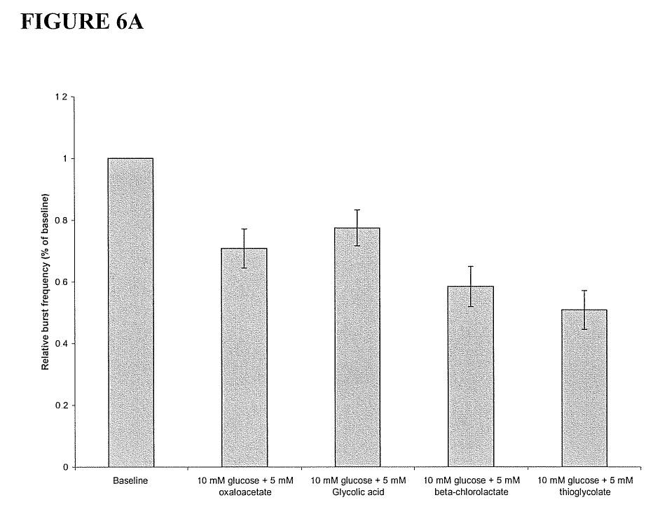

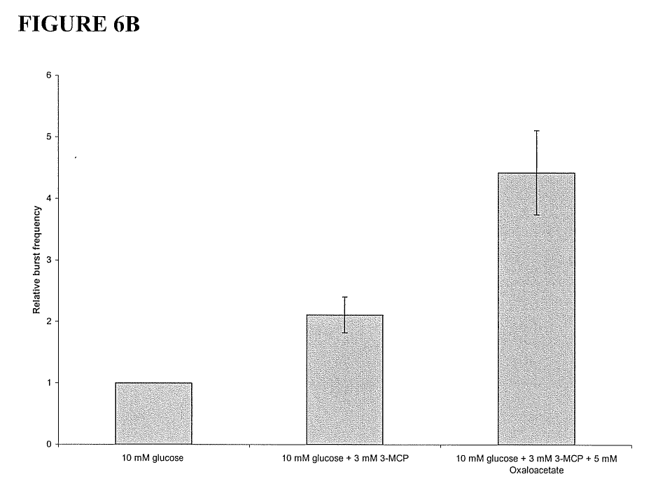

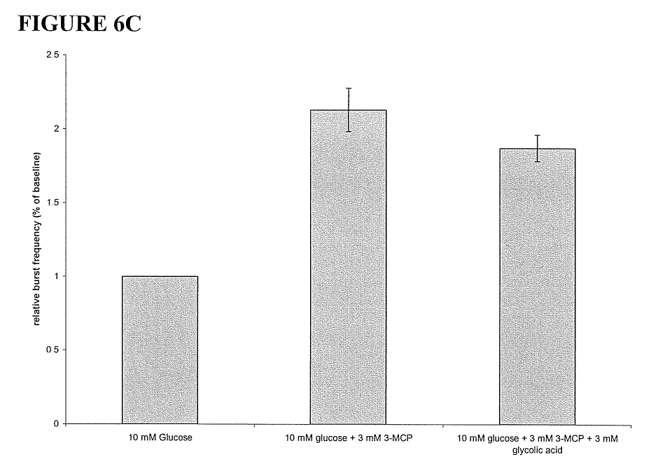

The effects of these alternative PEPCK substrates on synchronized burst discharges were examined in rat hippocampal slices ex corpora using the methods described in Example 1. FIGS. 6A through 6C confirm that it was indeed the rate of flux through PEPCK that was antiepileptic, as opposed to any effects downstream of PEPCK in the gluconeogenesis pathway. FIG. 6A shows that the usual substrate for PEPCK, oxaloacetate, as well as the alternative substrates, glycolic acid, β-chloroacetate, and thioglycolate, all reduce epileptiform bursting frequency in normal glucose conditions. FIG. 6B confirms that flux through PEPCK is essential for the reduction in epileptiform bursting, as the usual substrate for PEPCK, oxaloacetate, which causes a decrease in bursting under normal glucose conditions, cannot block the increase in bursting caused by inhibition of PEPCK by 3-MCP. FIG. 6C also shows that flux through PEPCK is important: although the PEPCK, substrate in FIG. 6C modestly blocks an increase in bursting caused by inhibition of PEPCK, glycolic acid is unable to cause the decrease in bursting that was demonstrated under normal glucose conditions (FIG. 6A) in the absence of a functional PEPCK. These results confirmed that metabolic modulation of flux through PEPCK affected the frequency of epileptiform bursting in hippocampal cell ex corpora, and indicated that such modulation would be capable of eliciting anti-seizure effects.

It should be understood that the foregoing disclosure emphasizes certain specific embodiments of the invention and that all modifications or alternatives equivalent thereto are within the spirit and scope of the invention as set forth in the appended claims.

What we claim is:

1. A method for reducing epileptic bursting in brain cells, the method comprising the step of contacting the cells with an effective amount of a compound that regulates the rate of flux through phosphoenolpyruvate carboxykinase (PEPCK).

2. The method of claim 1, wherein the compound increases the concentration of the PEPCK substrate oxaloacetate.

3. The method of claim 1, wherein the compound reduces the concentration of PEPCK reaction products.

4. The method of claim 1, wherein the compound is an alternative to the PEPCK substrate oxaloacetate.

5. The method of claim 3, wherein the compound is 2-deoxyglucose.

6. The method of claim 2, wherein the compound is oxaloacetate.

7. The method of claim 4, wherein the compound is glycolic acid, β-chloroacetate, L-glycerate or thioglycolate.

8. The method of claim 1, wherein the compound increases expression of a gene encoding PEPCK.

9. The method of claim 8, wherein the compound is glucagon, long-chain unsaturated fatty acids, oleate, dexamethasone, clofibrate, isoprenaline or retinoic acid.

10. The method of claim 1 wherein the brain cells are adult or juvenile brain cells.

11. A method for reducing synchronized bursting in neural cells, the method comprising the step of contacting the cells with an effective amount of a compound that regulates the rate of flux through phosphoenolpyruvate carboxykinase (PEPCK).

12. The method of claim 11, wherein the compound increases the concentration of the PEPCK substrate oxaloacetate.

13. The method of claim 11, wherein the compound reduces the concentration of PEPCK reaction products.

14. The method of claim 11, wherein the compound is an alternative to PEPCK substrate oxaloacetate.

15. The method of claim 13, wherein the compound is 2-deoxyglucose.

16. The method of claim 12, wherein the compound is oxaloacetate.

17. The method of claim 14, wherein the compound is glycolic acid, β-chloroacetate, L-glycerate or thioglycolate.

18. The method of claim 11, wherein the compound increases expression of a gene encoding PEPCK.

19. The method of claim 18, wherein the compound is glucagon, long-chain unsaturated fatty acids, oleate, dexamethasone, clofibrate, isoprenaline or retinoic acid.

20. The method of claim 11, wherein the neural cells are adult or juvenile neural cells.

21. A method for treating a seizure disorder in an adult or juvenile animal, the method comprising the step of administering an effective amount of a compound that regulates the rate of flux through phosphoenolpyruvate carboxykinase (PEPCK) to an animal in need thereof.

22. The method of claim 21, wherein the compound increases the concentration of the PEPCK substrate oxaloacetate.

23. The method of claim 21, wherein the compound reduces the concentration of PEPCK reaction products.

24. The method of claim 21, wherein the compound is an alternative to the PEPCK substrate oxaloacetate.

25. The method of claim 23, wherein the compound is 2-deoxyglucose.

26. The method of claim 22, wherein the compound is oxaloacetate.

27. The method of claim 24, wherein the compound is glycolic acid, □-chloroacetate, L-glycerate or thioglycolate.

28. The method of claim 21, wherein the compound increases expression of a gene encoding PEPCK.

29. The method of claim 28, wherein the compound is glucagon, long-chain unsaturated fatty acids, oleate, dexamethasone, clofibrate, isoprenaline or retinoic acid.

30. The method of claim 21, wherein the effect on the rate of flux through PEPCK occurs in adult or juvenile brain cells.

31. The method of claim 21 wherein the seizure disorder is epilepsy.

32. A pharmaceutical composition comprising a therapeutically-effective amount of a compound that regulates the rate of flux through phosphoenolpyruvate carboxykinase (PEPCK) and a pharmaceutically-acceptable excipient.

33. A pharmaceutical composition of claim 32, wherein the compound increases the concentration of the PEPCK substrate oxaloacetate.

34. A pharmaceutical composition of claim 32, wherein the compound reduces the concentration of PEPCK reaction products.

35. A pharmaceutical composition of claim 32, wherein the compound is an alternative to the PEPCK substrate oxaloacetate.

36. A pharmaceutical composition of claim 34, wherein the compound is 2-deoxyglucose.

37. A pharmaceutical composition of claim 33, wherein the compound is oxaloacetate.

38. A pharmaceutical composition of claim 35, wherein the compound is glycolic acid, β-chloroacetate, L-glycerate or thioglycolate.

39. A pharmaceutical composition of claim 34, wherein the compound is 2-deoxyglucose, 3-deoxy-D-glucose, 4-deoxy-D-glucose, 5-deoxy-D-glucose, 2, n-deoxy-D-glucose, where n=3-5, n, m deoxy-D-glucose, where n=2-5 and m=integers from 2-5 excluding n, sugars that can be metabolized into 2DG, such as 2-deoxy-D-galactose, halogenated and other conjugated derivatives of deoxy sugars, such as fluoro-2-deoxy-D-glucose, conjugated deoxy sugars that are metabolized to 2DG, and compounds having effects similar to 2DG, such as iodoacetate.

40. The method of claim 32, wherein the compound increases expression of a gene encoding PEPCK.

41. The method of claim 40, wherein the compound is glucagon, long-chain unsaturated fatty acids, oleate, dexamethasone, clofibrate, isoprenaline or retinoic acid.
